(19) United States
(12) Patent Application Publication
(10) Pub. No.: US 2026/0306569 A1
(43) Pub. Date: Oct. 8, 2026

(54) AGENT FOR IMPROVING VITREOUS FLOATERS AND METHOD FOR IMPROVING VITREOUS FLOATERS

(71) Applicant: PLUM Co., Ltd., Aichi (JP)

(72) Inventors: Itsuro KATSUDA, Aichi (JP); Kazuharu SUZUKI, Aichi (JP); Akio INOUE, Aichi (JP); Fumiya MIZUTANI, Aichi (JP); Tsuguyo YAMAZAKI, Aichi (JP); Yu INOUE, Aichi (JP); Motoko MIZUTANI, Aichi (JP)

(73) Assignee: PLUM Co., Ltd., Aichi (JP)

(21) Appl. No.: 19/480,595

(22) PCT Filed: Sep. 14, 2023
(86) PCT No.: PCT/JP2023/033620

§ 371 Date: Oct. 31, 2025

(51) Int. Cl. A61K 36/19; A61P 27/02

(52) U.S. Cl. CPC A61K 36/19; A61P 27/02; A61K 2236/15

(57) ABSTRACT

An agent for improving a vitreous floater containing Andrographis paniculata as an active ingredient has excellent safety and can be an agent for improving a vitreous floater which can prevent or treat a vitreous floater through daily intake.

### TECHNICAL FIELD

The present invention relates to an agent for improving a vitreous floater and a method for improving a vitreous floater.

### BACKGROUND ART

“Floaters” like worms, lint and the like sometimes seem to fly in front when staring at a bright place, a white wall, a blue sky or the like. The floaters seem to follow the movement of the eyes and do not disappear even by blinking or rubbing the eyes, but the floaters do not bother in a dark place. Such a symptom is medically called “vitreous floaters”.

The most part inside the eyeball is filled with a jelly-like transparent substance called the vitreous body, and light which enters through the cornea and the crystalline lens reaches the retina through the crystalline lens. When the vitreous body becomes cloudy for some reason, the shadows of the clouds are projected to the retina when looking at a bright place and sway with the movement of the eyeball, and this seems as if “floaters” like worms, lint and the like were flying, which are recognized as vitreous floaters.

A cause of the clouds of the vitreous body is an age-related change in the vitreous body. The composition of the vitreous body changes with age and degenerates into liquid from jelly, and a space in which liquid is accumulated is formed in the vitreous body and causes a cloud. Another possible cause of the clouds is detachment of the posterior surface from the adhesion with the retina (posterior vitreous detachment) through reduction in the volume of the vitreous body due to degeneration thereof. When the vitreous body degenerates as described above, the retina is pulled by the vitreous body with rapid eyeball movement, and the visual cells are thus stimulated, sometimes resulting in a symptom of photopsia, in which flashes of light appear in the sight. Although those who are in their early sixties frequently develop posterior vitreous detachment, the number of cases of vitreous floaters among young people has been recently increasing with an increase in the time spent using smart phones and the like.

To treat vitreous floaters, laser therapy for evaporating the vitreous opacities and cutting the vitreous strands by applying laser light, vitrectomy for removing the vitreous body in the eyeball with an instrument and the like are used, but the surgeries are rarely conducted actively because the surgeries are expensive and invasive.

However, because the vitreous opacities largely reduce the contrast sensitivity function, affect the eyesight and decrease the quality of life (QOL), a method which can improve vitreous floaters more easily with a lower burden is desired.

For example, an agent for improving a vitreous floater using a component derived from a plant of Curcuma of Zingiberaceae has been proposed (PTL 1).

### CITATION LIST

### Patent Literature

PTL 1: JP 2022-99937A

### SUMMARY OF INVENTION

### Technical Problem

A problem of the invention is to provide an agent for improving a vitreous floater which has excellent safety and which can prevent or treat a vitreous floater through daily intake.

### Solution to Problem

As a result of extensive studies to solve the problem, the present inventors have found that Andrographis paniculata has an excellent effect of improving a vitreous floater and thus have completed the invention.

That is, the invention relates to an agent for improving a vitreous floater, posterior vitreous detachment or photopsia containing Andrographis paniculata as an active ingredient.

Moreover, the invention relates to a method for improving a vitreous floater, posterior vitreous detachment or photopsia characterized by administering Andrographis paniculata to a human.

### Advantageous Effects of Invention

Andrographis paniculata which is the active ingredient of the agent for improving a vitreous floater of the invention has long been used as a herb for health maintenance or as a crude drug, is highly safe and can exert an excellent therapeutic/preventive effect on a vitreous floater through intake thereof.

### DESCRIPTION OF EMBODIMENTS

The agent for improving a vitreous floater of the invention contains Andrographis paniculata as an active ingredient.

Andrographis paniculata is a plant of Andrographis of Acanthaceae, is an annual plant which grows abundantly in southeast Asian countries such as India, Pakistan, Sri Lanka and Indonesia and which is 0.5 to 1 m tall with branches and leaves and contains diterpene lactone compounds such as andrographolide. The part of Andrographis paniculata used in the invention is not particularly restricted, and a leaf, a stem, a root, a flower, a seed, a fruit, the whole plant or the like can be used. Of these, leaves contain 2% by mass or more of andrographolide (a diterpene lactone), and thus a leaf is preferably used. Examples of the Andrographis paniculata as the active ingredient of the invention include the plant body itself, a squeeze obtained by pressing, a dried product obtained by drying the plant body, a pulverized product thereof, an extract extracted therefrom, and in view of the effect of improving a vitreous floater and the like, pulverized Andrographis paniculata is preferable.

The method for pulverizing Andrographis paniculata is not particularly limited, and an example thereof is a method of drying Andrographis paniculata, cutting into an appropriate size, then sterilizing and pulverizing with a pulverizing device such as a hammer mill, a mill, a mortar and millstones. The size of the Andrographis paniculata after pulverization is not particularly limited, and is, for example, around 1 mm to 1 μm, preferably around 1 mm to 10 μm.

The extraction method of Andrographis paniculata with a solvent is not particularly limited, and for example, extraction may be appropriately conducted by immersing Andrographis paniculata in water, an alcohol, a liquid oil or the like and extracting at normal temperature or the like.

The form of the agent for improving a vitreous floater of the invention is not particularly restricted, and the agent can be in the form of a pharmaceutical, a quasi-drug, a food or a drink, a supplement or the like for a human. For the pharmaceutical or the quasi-drug, Andrographis paniculata can be formulated directly or in combination with a pharmaceutically acceptable carrier according to the need. Furthermore, according to the need, a usual additive can also be appropriately added. The dosage form is not particularly limited, and a dosage form which can be orally taken is preferable because the effect can be exerted through oral intake. Examples thereof include capsules, a liquid preparation, powder, granules, and tablets. These can be produced according to a normal method.

In the case of a form of a food or a drink, the food or the drink can be prepared using Andrographis paniculata directly or by blending a known food additive or a food material as needed according to a normal method. The form thereof is not particularly restricted, and examples thereof include foods such as bread, cookies, cakes and noodles, confectionery such as gums, candies and Japanese confectionery, livestock products such as hams and sausages, fish products such as chikuwa (hollow cylindrical fish sausages) and kamaboko (steamed fish paste), fish and shellfish, seasonings such as dressing, soy sauce, jam and furikake (dry Japanese seasonings), drinks such as tea, juice, soft drinks and alcohols, functional foods in the same forms as those of the pharmaceutical or the like described above (capsules, a liquid preparation, powder, granules or tablets), foods for specified health uses, and foods with function claims.

The agent for improving a vitreous floater of the invention explained above can improve a symptom of a vitreous floater through administration to a mammal including a human, and a floater in the sight can be reduced or can disappear compared to that before the intake. The method for administering the agent for improving a vitreous floater of the invention is not particularly limited, but is preferably oral administration because the effect can be exerted through oral administration. In the invention, the agent for improving a vitreous floater includes use for both treating and preventing a vitreous floater. Moreover, as described above, posterior vitreous detachment and photopsia are closely related to vitreous floaters, and the invention also includes use for improving (preventing/treating) posterior vitreous detachment and/or photopsia.

The agent for improving a vitreous floater, posterior vitreous detachment or photopsia of the invention can have a label of, for example, preventing and treating vitreous floaters, preventing and treating posterior vitreous detachment, preventing and treating photopsia, improving the contrast sensitivity, or clearing the sight. Examples of the labeling act include describing on the main part, a container, packaging or the like of a product, describing on a document which discloses the product information such as an instruction manual, an attached document, a brochure and other prints, and describing on an advertisement used for advertising including flyers and the Internet.

When the agent for improving a vitreous floater of the invention is administered to a mammal, for example a human, the amount per administration is 1200 mg or more in terms of Andrographis paniculata (21.6 mg or more in terms of andrographolide), preferably 1200 to 2400 mg (21.6 to 43.2 mg in terms of andrographolide), and the amount is administered three times, namely morning, noon and evening. The amount per day is preferably 3600 mg or more in terms of Andrographis paniculata (64.8 mg or more in terms of andrographolide), preferably 3600 to 7200 mg (64.8 to 129.6 mg in terms of andrographolide). This is preferably taken continuously at least for five days or longer, preferably 14 days or longer, preferably one month or longer.

### EXAMPLES

The invention will be explained in detail below referring to Examples, but the invention is not limited to the Examples.

### Production Example 1

Production of Andrographis paniculata Capsules:

Leaves of Andrographis paniculata were cut into an appropriate size, sterilized and then pulverized with a hammer mill into around 100 to several hundred micrometers, and fine powder of Andrographis paniculata leaves was thus obtained. The andrographolide content of the fine powder of Andrographis paniculata leaves which was measured by HPLC was 5.4% by mass. Size 1 transparent hard capsules (made of gelatin) were filled with 300 mg of the fine powder of Andrographis paniculata leaves and sealed using hardened rapeseed oil according to a normal method, and thus Andrographis paniculata capsules were produced.

### Example 1

### Vitreous Floater Improvement Test:

Patients A to F having vitreous floaters took five Andrographis paniculata capsules obtained in Production Example 1 each in the morning, at noon and in the evening for two to four months. The diagnosis and examination results by ophthalmologists or the subjective assessment of the patients before and after the intake of the capsules are shown.
(1) Patient A
   Before Intake:
      The patient had mild vitreous floaters and felt vitreous floaters when looking at a white wall. When the patient was examined by an ophthalmologist, the patient was diagnosed with vitreous floaters.
   After Intake:
      When the patient was examined by an ophthalmologist after one month of intake, it was found that the vitreous floaters were still observed but became considerably weaker. The patient felt that the vitreous floaters (floaters) disappeared after four months of intake.
(2) Patient B
   Before Intake:
      The patient already had vitreous floaters and had been receiving therapy for two years, but no improvement was observed. When the patient was examined by an ophthalmologist, the patient was diagnosed with vitreous floaters.
   After Intake:
      The patient felt that the vitreous floaters (floaters) disappeared after two months of intake. When the patient was examined by an ophthalmologist after three months of intake, no vitreous floaters were found.
(3) Patient C
   Before Intake:
      When the patient was examined by an ophthalmologist, the patient was diagnosed with vitreous floaters.
   After Intake:
      The patient no longer saw vitreous floaters (floaters) in one month with intake.
(4) Patient D
   Before Intake:
      The patient suffered from vitreous floaters for around 10 years. When the patient was examined by an ophthalmologist, the patient was diagnosed with vitreous floaters.
   After Intake:
      The vitreous floaters became weak after two months of intake, and the sight became clear. The patient no longer saw vitreous floaters (floaters) in four months with the intake.
(5) Patient E
   Before Intake:
      The patient suffered from vitreous floaters for around five years. When the patient was examined by an ophthalmologist, the patient was diagnosed with vitreous floaters.
   After Intake:
      When the patient was examined by an ophthalmologist after two, four and six months of intake, it was found each time that there was no problem. The patient no longer saw small black shadow-like things which moved slowly and black things like shooting stars which the patient used to see when moving the eyes largely after two months of intake.
      As described above, when the patients who were diagnosed with vitreous floaters took Andrographis paniculata, the vitreous floaters were improved.

### INDUSTRIAL APPLICABILITY

The agent for improving a vitreous floater of the invention can be used as a medicine, a food or a drink or the like for improving a vitreous floater.

1-6. (canceled)

7. A method for improving a vitreous floater comprising
administering a composition comprising Andrographis paniculata as an active ingredient to a human in need thereof.

8. The method of claim 7, wherein the Andrographis paniculata is selected from the group consisting of a plant body of Andrographis paniculata, a squeezed product obtained by pressing Andrographis paniculata, a dried product obtained by drying the plant body of Andrographis paniculata, a pulverized product of Andrographis paniculata, and an extract extracted from Andrographis paniculata.

9. The method of claim 7, wherein the Andrographis paniculata is in the form of a pulverized product of Andrographis paniculata.

10. The method of claim 9, where a size of the Andrographis paniculata after pulverization in the range of 1 mm to 10 μm.

11. The method of claim 7, wherein the Andrographis paniculata is administered orally.

12. The method of claim 7, wherein the amount of Andrographis paniculata per administration is 1200 mg to 2400 mg.

13. The method of claim 12, wherein the amount of Andrographis paniculata administered per day is 3600 mg to 7200 mg.

14. The method of claim 13, wherein the Andrographis paniculata is administered for at least 14 continuous days.
